(19) United States
(12) Patent Application Publication
(10) Pub. No.: US 2025/0336550 A1
(43) Pub. Date: Oct. 30, 2025

(54) AI-Driven Care Planner with Enhanced Personalization and Incentive Management

(71) Applicant: Healthcare Interactive, Inc., Glenwood, MD (US)

(72) Inventors: Henry H. Cha, Glenelg, MD (US); Xavier Cha, Glenwood, MD (US); Alexander Straub, Glenwood, MD (US)

(21) Appl. No.: 18/885,730

(22) Filed: Sep. 15, 2024

Related U.S. Application Data
(63) Continuation-in-part of application No. 18/652,797, filed on May 1, 2024.
Application No. 18/652,797 is a continuation-in-part of application No. 18/650,875, filed on Apr. 30, 2024, now abandoned.

(51) Int. Cl. G16H 80/00; G16H 20/00

(52) U.S. Cl. CPC G16H 80/00; G16H 20/00

(57) ABSTRACT

A system for automated healthcare management of a patient including a virtual agent system that includes multiple specialized personas, each configured to perform specific tasks within a healthcare management process. The personas include a schedule builder persona configured to generate a personalized care plan based on patient data, integrating input from one or more expert resources to tailor the care plan to the patient's needs; and a message builder persona configured to generate and schedule patient communications based on the generated personalized care plan, ensuring that each message is compliant with healthcare regulations.

### CROSS-REFERENCE TO RELATED APPLICATIONS

This application is a continuation-in-part of U.S. application Ser. No. 18/652,797, filed May 1, 2024, and entitled “Virtual Agent,” which is a continuation-in-part of U.S. application Ser. No. 18/650,875, filed Apr. 30, 2024, and entitled “Virtual Agent.” The entire disclosure of each application mentioned above is incorporated herein by reference.

### TECHNICAL FIELD

The present disclosure generally relates to virtual agents and, more particularly, to virtual agents with customizable personas and stackable resources.

### BACKGROUND

Virtual agents are computer-generated agents that can interact with users. Virtual agents may communicate with human users in a natural language and work with or otherwise assist users with the performance of various tasks, such as information retrieval and obtaining rule-based recommendations. Informally, virtual agents may be referred to as “chatbots.” Virtual agents may be used by corporations to assist customers with tasks such as retrieving membership or benefits information. Using virtual agents may offer a corporation advantages by reducing operational costs of running call centers.

Traditional care management systems often lack the ability to provide personalized and adaptive care plans tailored to individual user needs. There is, therefore, a need for more sophisticated systems that can dynamically adjust to user behavior, integrate health data, and provide personalized insurance recommendations.

There is a need in the art for a system and method that addresses the shortcomings discussed above.

### SUMMARY

The present disclosure is directed to virtual agents with customizable personas and stackable resources. The present disclosure further describes the integration of new personas and expert resources that deliver a more comprehensive and responsive care management solution. In particular, an AI-driven care planner is disclosed that includes a schedule builder persona for creating personalized care plans, a message builder persona for crafting guideline compliant communications, and a feedback collector persona for dynamically adjusting care plans based on user input. Additionally, the system integrates real-time data to provide tailored insurance product recommendations, further personalizing the user experience and ensuring that care plans are both adaptive and comprehensive.

In one aspect, the present disclosure is directed to a system for generating personalized healthcare schedules within a virtual agent system. The system includes a device processor and a non-transitory computer readable medium having stored thereon instructions, executable by the processor, for performing the following steps: receiving patient data regarding a patient; and utilizing a schedule builder persona within the virtual agent system to create a care plan that is tailored to the patient's needs, has a predetermined duration, and includes details regarding timing and content of communication to be made with the patient.

In another aspect, the present disclosure is directed to a system for generating personalized healthcare messaging within a virtual agent system. The system includes a device processor and a non-transitory computer readable medium having stored thereon instructions, executable by the processor, for performing the following steps: receiving patient data regarding a patient; and utilizing a message builder persona within the virtual agent system to generate a series of personalized messages based on the created care plan.

In another aspect, the present disclosure is directed to a system for directing a patient to healthcare programs within a virtual agent system. The system includes a device processor and a non-transitory computer readable medium having stored thereon instructions, executable by the processor, for performing the following steps: receiving patient data regarding a patient; and utilizing an healthcare program specialist persona to navigate prescription drug programs and identify suitable options for the patient based on the patent's current medications and medical conditions.

In another aspect, the present disclosure is directed to a system for automated healthcare management of a patient. The system includes a virtual agent system that includes multiple specialized personas, each configured to perform specific tasks within a healthcare management process. The personas include a schedule builder persona configured to generate a personalized care plan based on patient data, integrating input from one or more expert resources to tailor the care plan to the patient's needs; and a message builder persona configured to generate and schedule patient communications based on the generated personalized care plan, ensuring that each message is compliant with healthcare regulations. The system is also configured to consult multiple specialized expert resources for analysis of patient data and available healthcare programming. The expert resources include a feedback expert resource configured to receive and analyze real-time feedback from the patient and provide insights that allow the schedule builder persona and message builder persona to adjust the generated care plan and the generated patient communications; and an incentive management expert resource configured to monitor patient compliance with health plan activities set forth in the generated care plan and incorporating user-specific goals and rewards into the care plan.

In another aspect, the present disclosure is directed to a method of self-learning generative artificial intelligence (AI) prompt optimization. The method may include utilizing a device processor to execute instructions stored on a non-transitory computer readable medium for performing the following steps: establishing an input prompt; generating care plan messages using the input prompt; using a prompt engineering insights expert resource to determine a prompt output score; determining whether the prompt output score meets a predetermined scoring threshold; and recording the input prompt as an optimized prompt if the prompt output score meets the predetermined scoring threshold and generating a new input prompt if the prompt output score does not meet the predetermined scoring threshold.

In another aspect, the present disclosure is directed to a method of omnichannel communication. The method may include a utilizing a device processor to execute instructions stored on a non-transitory computer readable medium for performing the following steps: receiving patient data regarding a patient; receiving user input from the patient via a virtual agent; and, based on the received patient data and user input, implementing an output manager expert resource to generate a plurality of omnichannel communications to be sent to the patient.

Other systems, methods, features, and advantages of the disclosure will be, or will become, apparent to one of ordinary skill in the art upon examination of the following figures and detailed description. It is intended that all such additional systems, methods, features, and advantages be included within this description and this summary, be within the scope of the disclosure, and be protected by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like reference numerals designate corresponding parts throughout the different views.

FIG. 1 is a schematic diagram of architecture for a virtual agent that corresponds with a user;

FIG. 2 is a schematic diagram of additional details of the virtual agent architecture;

FIG. 3 is a schematic diagram of an initialization process of a virtual agent with customizable personas;

FIG. 4 is a schematic diagram of an embodiment of a reinforcement learning process;

FIG. 5 is a schematic diagram of an embodiment of a virtual agent comprising a Deep Q Network process for learning;

FIG. 6 is a schematic diagram of an embodiment of a Deep Q Network process for learning in the context of a virtual agent;

FIG. 7 is a schematic diagram of the role of a persona in the context of the operation of a virtual agent;

FIG. 8 is a schematic diagram illustrating a process of proactively retrieving data from multiple knowledge expert resources and providing information to a user based on the retrieved data;

FIG. 9 is a schematic block diagram of a system for generating personalized healthcare schedules within a virtual agent system;

FIG. 10 is a more detailed block diagram of the system shown in FIG. 9;

FIG. 11 is a schematic illustration of a feedback collection interface associated with a feedback collection persona utilized by the system of FIGS. 9 and 10;

FIG. 12 is a schematic illustration of a pop-up window providing personalized wellness tips based on user feedback received at the interface shown in FIG. 11;

FIG. 13 is a schematic dataflow diagram illustrating the implementation of a message builder persona;

FIG. 14 is a schematic data workflow illustrating the implementation of a healthcare program specialist persona;

FIG. 15 is a flowchart illustrating a method of implementing a schedule builder persona;

FIG. 16 is a flowchart illustrating a method of implementing a message builder persona;

FIG. 17 is a flowchart illustrating a method of implementing a healthcare program specialist persona;

FIG. 18 is a schematic diagram of an embodiment of a self-learning generative AI prompt optimization process; and

FIG. 19 is a flowchart illustrating a method of omnichannel communication.

### DESCRIPTION OF EMBODIMENTS

The present disclosure is directed to virtual agents with customizable personas and stackable resources. The persona of the virtual agent may be customizable between various emotional approaches. In addition, the virtual agent may be configured to retrieve information from various stackable resources. These stackable resources are referred to herein as knowledge experts or knowledge expert resources. These resources may comprise databases or repositories of information which is organized and categorized to facilitate accurate retrieval of information without overloading the user with responses that include extraneous information that is not relevant to the user's inquiry.

The disclosed virtual agent system may be configured to access these resources in a tiered approach. For example, the virtual agent may refer to a first knowledge expert resource based on the initial inquiry by the user. Then, based on information retrieved from the first knowledge expert resource, the system may choose a second knowledge expert resource to consult. For instance, if a user requests insurance benefits information regarding a given insurance claim, the system will first access a knowledge expert resource that includes information about the claim in question. Then, depending on what medical procedure the claim is for, the system will access a select second knowledge expert to retrieve the user's insurance benefits/coverage for the medical procedure to which the insurance claim is directed.

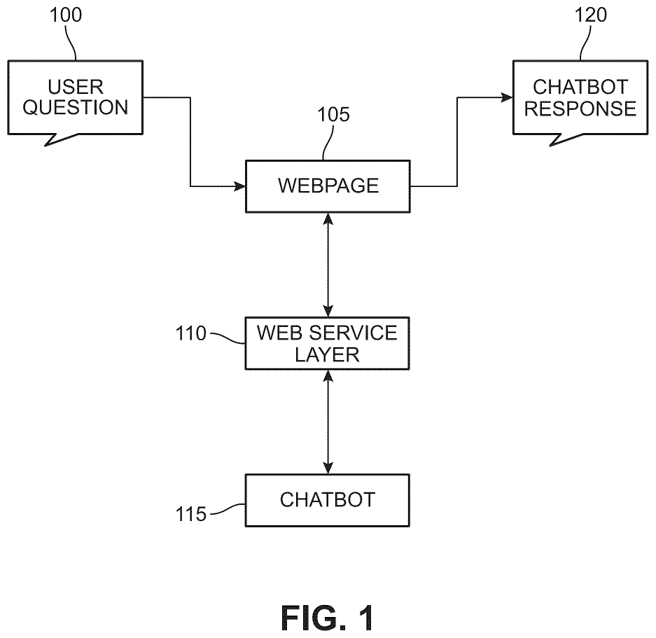

FIG. 1 is a schematic diagram of architecture for a virtual agent that corresponds with a user. As shown in FIG. 1, the user presents a question at step 100. In some embodiments the disclosed virtual agent may communicate with a customer via text-based communication (e.g., SMS or a chat-based application). That is, the virtual agent may be a so-called “chatbot.” In some embodiments, it is also possible that the disclosed virtual agent may communicate with the user via a video platform.

A virtual agent may include additional subsystems and modules to achieve the goal of conversing with a user. For example, an end user communicates with virtual agent through various modes, including text-based chat programs that may run on a desktop, laptop or mobile device, telephone calls, audio, and/or video calls transmitted over the internet, as well as other known modes of communication.

A virtual agent and associated systems for communicating with a virtual agent may include one or more user devices, such as a computer, a server, a database, and a network. For example, a virtual agent running on a server could communicate with a user over a network. In some embodiments, the network may be a wide area network (“WAN”), e.g., the Internet. In other embodiments, the network may be a local area network (“LAN”). For example, in a more remote location far from a metropolitan area, the Internet may not be available. In yet other embodiments, the network may be a combination of a WAN and a LAN. In embodiments where a user talks to a virtual agent using a phone (e.g., a landline or a cell phone), the communication may pass through a telecom network and/or a wide area network.

The user device may be a computing device used by a user for communicating with a virtual agent. A computing device could be a tablet computer, a smartphone, a laptop computer, a desktop computer, or another type of computing device. The user device may include a display that provides an interface for the user to input and/or view information. For example, a user could interact with a virtual agent using a program run on a laptop computer, such as a text-based chat program, a voice-based communication program, and/or a video-based communication program. Alternatively, in some cases, the user device could be a telephone (e.g., a landline, cell phone, etc.).

One or more resources of a virtual agent may be run on one or more servers. Each server may be a single computer, the partial computing resources of a single computer, a plurality of computers communicating with one another, or a network of remote servers (e.g., cloud). The one or more servers can house local databases and/or communicate with one or more external databases.

As also shown in FIG. 1, the user question input is made via a software platform, such as a webpage 105. Webpage 105 may then access a web service layer 110 and ultimately a chatbot intelligence center 115. The chatbot may process the user's question, retrieve the relevant data and deliver the data back to webpage 105 via web service layer 110. Then, the system may produce a chatbot response 120 to the user.

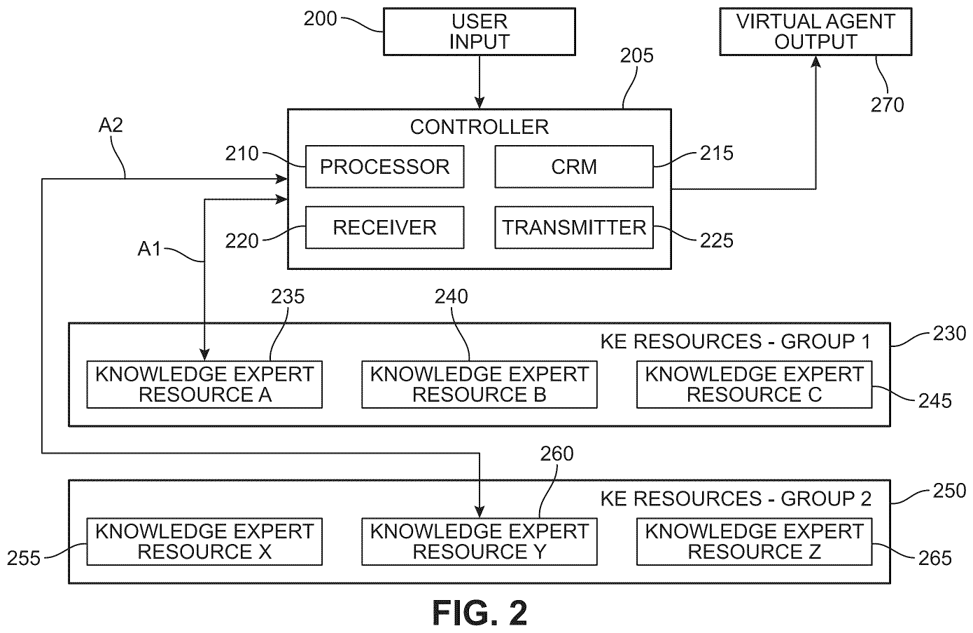

FIG. 2 is a schematic diagram of additional details of the virtual agent architecture. As shown in FIG. 2, a user may submit input 200, typically an inquiry/request for information. This user input 200 is received by the virtual agent system, particularly by a controller 205.

Controller 205 may include various computing and communications hardware. For example, as shown in FIG. 2, controller 205 may include a device processor 210 and a non-transitory computer readable medium 215 including instructions executable by device processor 210. Computer readable medium 215 may include any suitable computer readable medium, such as a memory, e.g., RAM, ROM, flash memory, or any other type of memory known in the art. Controller 205 may include other computing hardware, such as servers, integrated circuits, displays, etc.

Further, controller 205 may include networking hardware configured to interface with other nodes of a network, such as a LAN, WLAN, or other networks. For example, as shown in FIG. 2, controller 205 may include a receiver 220 and a transmitter 225. (It will be appreciated that, in some embodiments, the receiver and transmitter may be combined in a transceiver.) Receiver 220 and transmitter 225 may be configured to provide communication with other nodes of the system. Such communication may be executed via any suitable format, such as satellite communication, radiofrequency signals, etc.

Controller 205 may be provided at any suitable location. In some cases, controller 205 may be provided at a headquarters of a service provider. In other cases, controller 205 may be provided at a dedicated host facility configured to coordinate the virtual agent processes.

Computer readable medium 215 of controller 205 may include instructions for receiving a user request for one or more services. For example, as shown in FIG. 2, controller 205 may be configured to receive requests from users accessing the system via various access tools. For example, some users may access the system via the Internet, e.g., with a laptop. Some users may access the system via an application (app) on a personal electronic device, such as a smart phone. These users may submit requests for information using these access tools.

In some embodiments, the user input may be in the form of text communication. In some embodiments, the user input may be in the form of verbal communication. In some embodiments, the user may provide input via either text or verbal input. In some embodiments, the user input may be provided via a video interface.

As also shown in FIG. 2, the virtual agent may be configured to retrieve information from various stackable resources. That is, an intent recognition system of the disclosed system may be configured to determine the selection of which knowledge expert resource(s) to consult and in which order. For example, as shown by a first arrow A1 in FIG. 2, upon receiving a user input including a request for information, the system may direct a first query to a first knowledge expert resource based on the request for information. In some embodiments, the first knowledge expert resource may be selected from a plurality of knowledge expert resources. For example, the first knowledge expert resource may be selected from a first group of knowledge expert resources 230. As shown in FIG. 2, for example, knowledge expert (KE) resources group 1 (230) may include multiple resources, such as knowledge expert resource A (235), knowledge expert resource B (240), knowledge expert resource C (245), and any further number of resources suitable for the given category/type of information stored therein. As an example, FIG. 2 shows the system directing the first query to knowledge expert resource A (235) and, as indicated by double-headed arrow A1, controller 205 may receive a first set of data back from knowledge expert resource A (235).

In addition, as indicated by a second arrow A2, the system may direct a second query to a second knowledge expert resource based on the first set of data received from the first knowledge expert resource. In some embodiments, the second knowledge expert resource may be selected from a plurality of knowledge expert resources. For example, the second knowledge expert resource may be incorporated into a second group of resources, e.g., KE resources group 2 (250), which may include knowledge expert resource X (255), knowledge expert resource Y (260), knowledge expert resource Z (265), and any further number of resources. As shown in FIG. 2, the system may send the second query to knowledge expert Y based on the first set of data received from knowledge expert resource A. The system may pass along some or all of the information (e.g., context and/or insights) received from the first consulted knowledge expert resource A (235) to the second consulted knowledge expert resource Y (260). Accordingly, the information received from second consulted knowledge expert resource Y (260) may be stacked with the information received from the first consulted knowledge expert resource A (235).

It will be understood that, although two knowledge expert resources are accessed in the example provided in FIG. 2, the system may access any number of knowledge expert resources. That is, the system may direct queries to as many layers/tiers of knowledge expert resources as necessary to retrieve the data required to answer the user's inquiry.

Controller 205 may be further configured to receive a second set of data from the second knowledge expert resource (in this case knowledge expert resource Y), as indicated by double-headed arrow A2. Finally, based on the second set of data, the system may deliver, to the user, a response to the first request for information (i.e., virtual agent output 270).

The first knowledge expert resource is selected based on intent recognition of the system whereby intent of the user is recognized by the system. In some embodiments, the system may be configured to learn intent of users. In addition, in some embodiments the system may be configured to learn intent of users in real time based on user feedback. For example, after providing an output to the user, the system may question the user to determine the accuracy/usefulness of the output. In some cases, the virtual agent may question the user with a simple query, such as “Was this information helpful?” If the user answers “yes,” then the system can decipher that the system's assessment of the user's intent was correct and remember to make the same assessment when receiving the same or similar user input in the future. If the user answers “no,” then the system can decipher that the system's assessment of the user's intent was incorrect and may avoid making the same assessment in the future. Over time and across many user interactions, the virtual agent may improve its intent recognition.

The system may also base its selection of the second knowledge expert resource on intent recognition. As with selection of the first knowledge expert resource, the system's intent recognition with respect to the selection of the second knowledge expert resource may be learned, in some cases based on user feedback. The system may be configured to consult with various knowledge expert resources in a predetermined order based on the intent recognition. That is, the order in which queries are directed to different knowledge expert resources is based on predetermined instructions stored in the non-transitory computer readable medium. The system may include an intent composer module that is provided with instructions as to which knowledge expert resources to consult for a given user inquiry or type of inquiry, as well as instructions as to what order the multiple knowledge expert resources are to be consulted to retrieve accurate data upon which the base the chatbot's answer to the user's inquiry.

Regardless of how many knowledge expert resources are consulted in response to a given inquiry, it is the data retrieved from the last knowledge expert resource to be consulted that will most influence the answer to be provided to the user. For this reason, the order in which the knowledge experts are consulted is determinative as to whether the answer provided to the user is accurate and/or useful. It will also be understood that, sequential consultation of knowledge expert resources provides more accurate/useful answers than accessing multiple knowledge expert resources simultaneously. Again, the selection of the second knowledge expert resource to be consulted may be based, at least in part, on the data retrieved from a first knowledge expert resource to be consulted. Likewise, the selection of the third knowledge expert resource to be consulted may be based, at least in part, on the data retrieved from the second knowledge expert resource to be consulted, and so forth.

In some embodiments, the persona of the virtual agent may be customizable between various emotional approaches. For example, some users may prefer a soft touch, i.e., a more sensitive persona to interact with. Other users may prefer a more analytical approach, laying out the facts in a blunt and matter of fact manner. Some users may prefer brief responses from the personal agent, whereas other users may prefer more detailed responses. Therefore, in some embodiments, the persona of the virtual agent may be customizable between different emotional approaches, such as “soft touch,” analytical, brief, detailed, or other designated approaches. In some embodiments, the persona may be selectable by the user. In other embodiments, the persona may be auto-selected based on interaction with the user or information about the user.

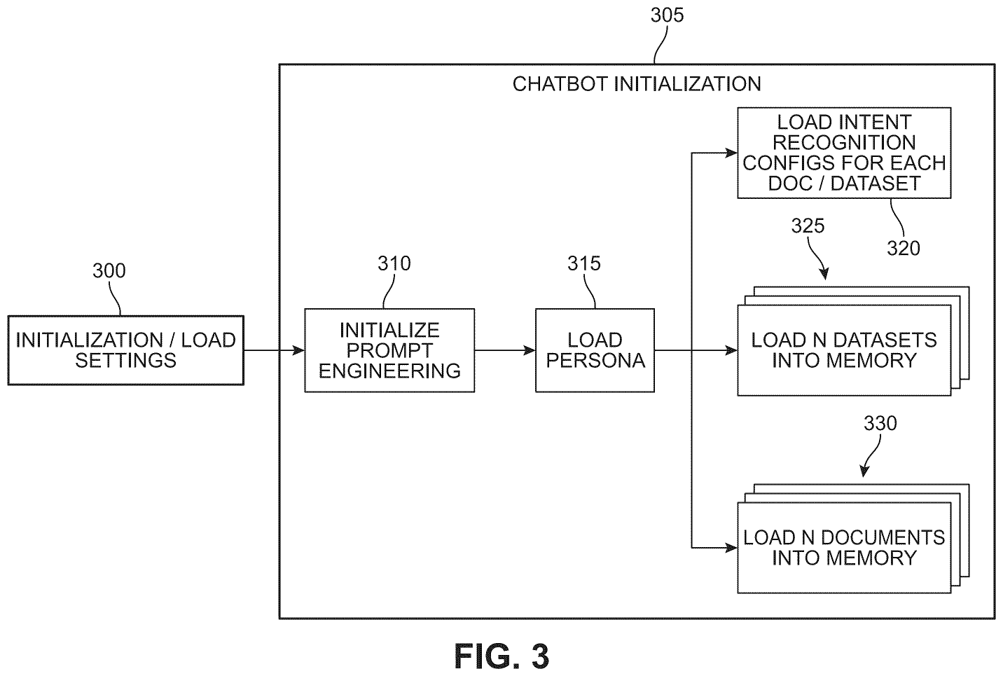

FIG. 3 is a schematic diagram of an initialization process of a virtual agent with customizable personas. As discussed above with respect to FIG. 2, the virtual agent system may include a device processor and a non-transitory computer readable medium having stored thereon instructions, executable by the processor. Via these instructions, the system may be configured to perform various tasks. As discussed above, the system may be configured to receive user input including a first request for information and, based on predetermined prompt engineering, direct a query to at least one knowledge expert resource based on the first request for information. In order to do so, the system may initialize/load various settings (300). The system may then commence chatbot initialization (305).

To initialize the chatbot, the system may initialize prompt engineering (310). The prompt engineering is the base set of instructions about how the chatbot will respond to various prompts. The system may include prompt engineering for one or more genres. For example, in some embodiments, the system may include prompt engineering for at least one of the following genres: finance, healthcare, and insurance. The non-transitory computer readable medium may include instructions for auto-selecting one of the listed genres based on the user input. In addition, the system may be configured to self-optimize the prompt engineering.

In addition to initializing the prompt engineering, the system also loads a persona (315). Various personas may include, for example, “analytical,” “soft touch,” “brief,” “detailed,” or other personas. An analytical persona may provide responses to the user that are very blunt or matter of fact. A “soft touch” persona may provide responses that are more sensitive to the emotions of the user. A brief persona may provide responses that are particularly concise, whereas a detailed persona may provide responses that provide more information/details to the user. It will be understood that the system may present one or more other types of personas with varying interactive characteristics.

In some embodiments, the type of persona may be selectable by the user. In some embodiments, the persona may be auto-selected by the system. In some cases, the persona may be auto-selected based on the user's input. For example, in order to auto-select a persona, the system may be configured to detect certain aspects about the user based on their input. Is the user extraordinarily polite or sensitive in their chat communication? If so, a “soft touch” persona may be auto-selected. At the other end of the spectrum, is the user particularly to-the-point or even crude with their communication? If so, an analytical persona may be auto-selected that provides feedback in a very matter of fact manner without much concern for the user's feelings. Is the user extremely brief with their communication? If so, a very brief and concise persona may be auto-selected. On the other hand, if the user is long-winded with their communication, a persona that provides very detailed responses may be auto-selected. It will be understood that various other types of personas may alternatively or additionally be selectable (or auto-selected).

In addition, the system is configured to load intent recognition configurations (320). That is, the intent recognition configurations are loaded from other databases into a server memory where the chatbot and personas are also hosted. The intent recognition configurations are parameters that govern how the system understands the user's questions and directs a relevant query to the correct data to retrieve useful information upon which to base a response to the user.

Further, the system is configured to load a predetermined number of datasets into memory (325). Similarly, the system may also be configured to load a predetermined number of documents into memory (330). As with the intent recognition configurations the documents and datasets are loaded from other databases into the server memory where the chatbot and personas are also hosted. Referring again to step 320, the system may load intent recognition configurations for each document and dataset that is loaded.

With the prompt engineering, persona, intent recognition configurations, datasets, and documents initialized/loaded, the system may process the user's inquiry and begin searching for information upon which to base a response.

In order to develop a virtual agent that can interact in a conversational manner with an end user, a reinforcement learning approach is utilized. Specifically, the virtual agent system uses deep reinforcement learning (DRL) to learn how to respond appropriately to user requests in a manner that is natural (i.e., human-like) and that meets the user's goals (e.g., retrieving insurance benefits information).

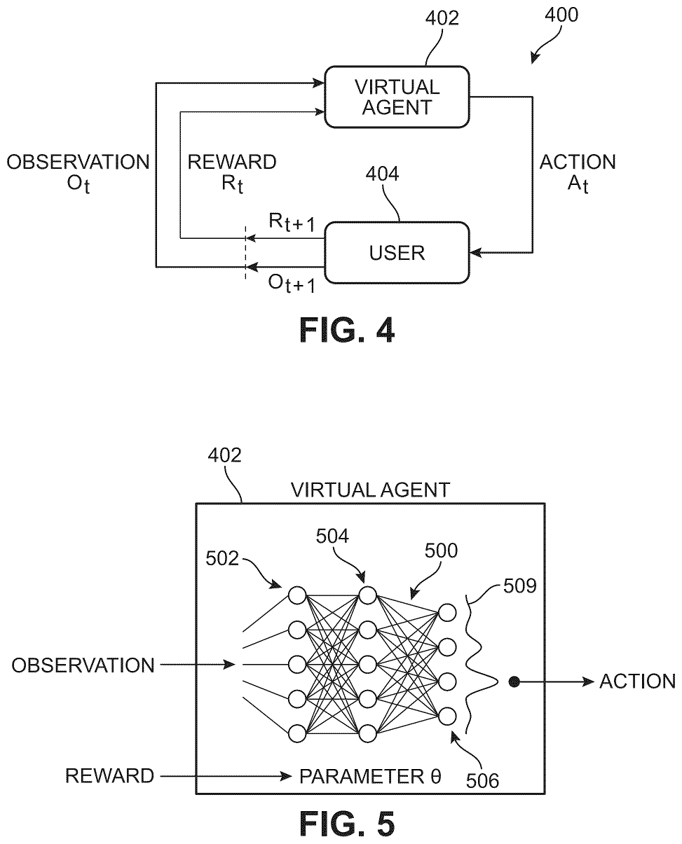

FIG. 4 is a schematic diagram of an embodiment of a reinforcement learning process. In particular, FIG. 4 is a schematic overview of a reinforcement learning system 400 for a dialogue management system. In a reinforcement learning system, a virtual agent 402 interacts with an external system or environment of some kind. In the context of dialoguing with an end user, user 404 may comprise the interacting environment.

The reinforcement learning system depicted in FIG. 4 is characterized by a repeating process: virtual agent 402 receives an observation Oₜ at time t of some aspect of the state of the system (i.e., of the user/environment). In some cases, the observation may be information related to the user's most recent response. In other cases, the observation could be part of, or the full, dialogue context. In still other cases, the observation could include other information gathered about the user, for example information from a video feed of the user that may capture nonverbal responses or gestures in addition to any verbal response. In addition to receiving an observation, virtual agent 402 may receive a reward Rₜ. In some cases, this reward could be explicitly provided by user 404 and/or the environment. In other cases, this reward could be determined by virtual agent 402 according to information from observation Oₜ, the dialogue context and/or any other information available to virtual agent 402 as it pertains to the state of the user/external system.

In response to receiving observation Oₜ, virtual agent 402 takes an action Aₜ at time t. The user responds to this action which generates a new observation Oₜ₊₁ at time t+1. An explicit or implicit reward Rₜ₊₁ may also be explicitly provided by the user or determined by virtual agent 402 using information about the user/external system. This process is repeated until learning is stopped. The virtual agent 402 learns which action A to take in response to an observation O by using the reward R as feedback to evaluate previous actions.

The learning characterized above is controlled by a particular reinforcement learning process. Some embodiments may employ a Q-Learning process, in which an agent tries to learn a function Q (s, a) that represents the “quality” of taking an action Aₜ=a in a state Sₜ=s. Thus, if an agent learns the correct Q function, they can choose an appropriate action in a state S by selecting the action A that yields the highest Q value for the current state. In the context of a dialogue management system, the state S is characterized by the virtual agent's observation O of the user's response and/or information such as the full dialogue context.

FIG. 5 is a schematic view of an embodiment of virtual agent 402 that is trained using a Deep Q Network (DQN) process. A Deep Q Network (DQN) is a Q-Learning system that uses one or more deep neural networks (DNNs) to learn the desired Q function for a given reinforcement learning task. For example, in FIG. 5, virtual agent 402 includes a deep neural network 500 (or simply, DNN 500). An input layer 502 of DNN 500 corresponds to the current observation (e.g., Oₜ of FIG. 4) of the system. Depending on the depth of the network, DNN 500 can include one or more intermediate or hidden layers (e.g., hidden layer 504). The nodes of output layer 506 each correspond to a particular Q value. Specifically, each node in the output layer 506 corresponds to a possible action (A₁, A₂, etc.) that may be taken in response to the current observation. That is, the values of the nodes in the output layer 506 correspond to the result of evaluating the Q-function (represented in FIG. 5 as function 509) in the context of a given observation O, for each possible action: Q (O, A₁), Q (O, A₂), etc. From the set of output Q values, the virtual agent selects the action corresponding to the largest Q value (i.e., the action associated with the node where function 509 is the largest). This action is then performed by the agent, and the reinforcement learning cycle shown in FIG. 4 is repeated.

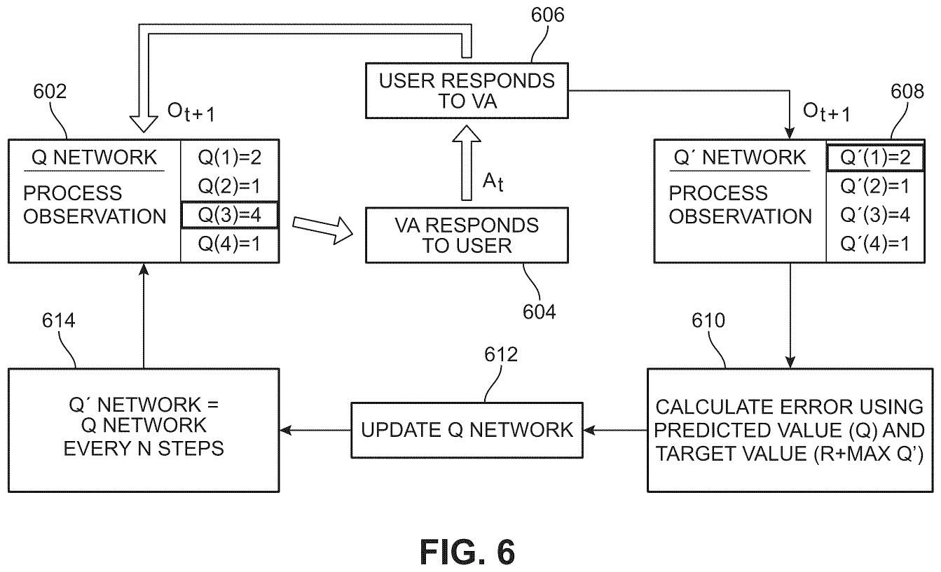

DNN 500 includes parameters (or weights) 0. During the learning process, the values of these parameters may be updated. As indicated schematically in FIG. 5, the values of these parameters depend on the rewards received during the training process. The specific relationship between the network parameters and the rewards is discussed in further detail below with respect to FIG. 6.

Although the description refers to training virtual agent 402, it may be appreciated that the learning processes described in this description may primarily occur within the dialogue management system, as it is the dialogue management system that ultimately makes decisions about what actions a virtual agent will take in response to cues from the user/environment.

FIG. 6 is a schematic view of some of the steps of the DQN training process. For clarity, some steps are not depicted here. Moreover, some of the steps have been greatly simplified.

The training process starts with the system in a known state. In the context of a dialogue management system, the state may be characterized by an observation of a user response in response to a known virtual agent action (such as a greeting). Alternatively, in some cases, the state could be characterized by a partial or full history of the dialogue. To obtain both the initial and subsequent user responses to future virtual agent actions, the dialogue management system may sample one or more training dialogues that comprise transcripts or simulations of conversations between a virtual agent and an end user.

The exemplary process of FIG. 6 may begin at a step 602, where a first DNN, referred to as the “Q Network,” may process an observation (e.g., an observation Oₜ, which is not shown). Here, “processing” the observation means feeding the observation (e.g., a user input/response) into the Q Network and making a forward pass through the network to output a set of Q values. The action associated with the largest Q value is selected, and the agent performs this action Aₜ at a step 604. In turn, the user responds to the virtual agent in a step 606, which changes the state of the user/environment and generates a new observation Oₜ₊₁. As discussed, the user response may be determined by a set of training dialogues. After the user has responded, the system may also generate a reward Rₜ₊₁ to be used later in subsequent steps.

To facilitate learning, the Q Network needs to be updated before the agent takes an action in response to the new observation Oₜ₊₁. To update the Q Network, the error between the outputs of the Q Network and their expected values must be calculated. In the DQN system, the expected values are determined by a target function. The target function (F_{TARGET}) is a function of the reward R (if any) received after taking the last action and of the Q function evaluated on the new observation. Formally, F_{TARGET}=Rₜ+γ maxₐ Q (Oₜ, a). Here, γ is a learning “discount” factor, and the “max” operator is equivalent to calculating the value of Q for all possible actions and selecting the maximum value. The error is then calculated as the mean-squared-error between Q and F_{TARGET}. In practice, the DQN system uses a second “target” network, denoted Q′, to calculate F_{TARGET}. Q′ may be identical to Q, but may have different weights at some time steps in the training process. Using a separate target network, Q′ has been shown to improve learning in many contexts.

Therefore, to update the Q Network, a second (or target) DNN (denoted the “Q′ Network” in FIG. 6) is used to process the new observation and output a set of values Q′ (Oₜ, A₁), Q′ (Oₜ, A₂), etc., during a step 608. The maximum of these values is used, along with the reward R generated earlier, to calculate the target function. Then, the error for the Q Network is determined as a function of its outputs (Q values) and the target function in a step 610. In a step 612, the Q Network is updated via backpropagation using the error computed in step 610.

The Q′ Network is not updated during each training pass. Instead, every N steps, where N is a meta-parameter of the training process, parameters θ′ of the Q′ Network are set equal to the latest values of the parameters θ of the Q Network. That is, every N steps, the Q′ Network is simply replaced with a copy of the Q Network during a step 614.

Some embodiments may employ other techniques that can facilitate learning with DQNs. These can include the use of epsilon-greedy strategies and experience replay as well as other known techniques.

In some embodiments, a double deep Q learning (DDQN) system may be used. The DDQN system may be similar to the DQN system with some differences. In DDQN, the first DNN (e.g., the Q Network shown in FIG. 6) may be used to determine the best action at each training step, while the second DNN (e.g., the Q′ Network) may be used to determine the associated Q value for taking that action. In some cases, using DDQN may help reduce overestimation of Q values that can occur with a DQN process.

Other embodiments could employ still further variations in the DQN architecture. For example, in another embodiment, a dueling DQN architecture could be employed.

It may be appreciated that the reinforcement learning process may be used in some contexts, but not others. For example, in one embodiment, the reinforcement learning process described above may be used while the dialogue management system is trained, but the process may not be used when the dialogue management system is tested (and/or deployed for use with real end users). Thus, in the training phase, the system may generally operate according to the process depicted in FIG. 6. However, during the testing and/or deployment phases, only the processes indicated in step 602, step 604, and step 606 may be used (as indicated by the fatter arrows in FIG. 6), while the other steps (i.e., step 608, step 610, step 612, and step 614) are not used (as indicated by the thinner arrows in FIG. 6). In terms of the Q Network, during training, both forward and backward passes are made through the network. During testing/deployment, however, only forward passes are made to generate recommended actions.

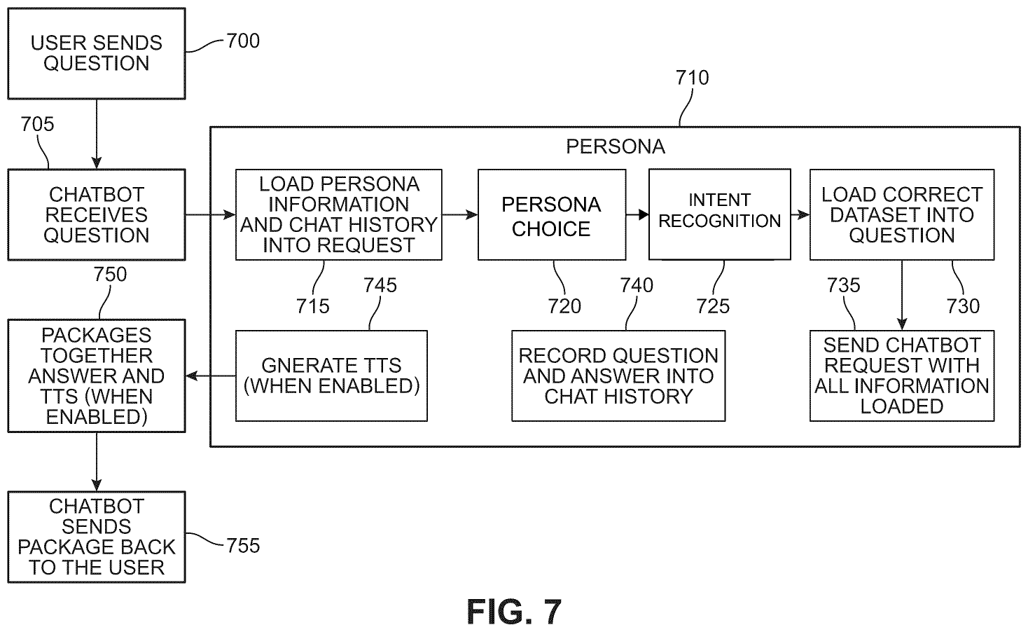

As discussed above, the persona with which the virtual agent communicates may be customizable. FIG. 7 is a schematic diagram of the role of a persona in the context of the operation of a virtual agent. As shown in FIG. 7, first, the user inputs a question to the system (700), i.e., an input into the chatbot including a first request for information. The chatbot receives the user input accordingly (705). The system then initializes a persona (710) with which to operate and loads persona information (and chat history information) into the request (715). At 725, the selection of a persona is made. Next, the system initializes intent recognition (720). In some embodiments, the intent recognition module varies based on the chosen persona. Again, this selection may be by user choice or it may be automated.

In addition, based on the intent recognition, the system loads the correct dataset (730). Then, the system sends the chatbot request to a given resource (735). That is, the system directs the query to at least one knowledge expert resource based on the first request for information input by the user. Upon consulting with the knowledge expert resource(s), the system receives a set of data from the knowledge expert resource(s), generates an answer based on the relevant information found with the resource, and records the question and answer into the chat history (740).

In some embodiments, the system may have Text To Speech (TTS) capability. When TTS is enabled, the system generates a TTS output for the answer to the user's question (745). The system then packages the answer and TTS (when enabled) together (750), and the chatbot delivers a response to the user by sending the package back to the user (755) in text or audio speech (if TTS is enabled) form.

As discussed above, the persona may selectable by a user or auto-selected. In some embodiments, the persona may be auto-selected based on the user input. In other embodiments, the persona may be auto-selected based on user information. For example, the persona may be auto-selected based on user information such as age, gender, occupation, or other information/characteristics of the user.

In some embodiments, the persona may be autogenerated based on the user input. In order to autogenerate the persona, the system may utilize two artificial intelligence (AI) bots running simultaneously. One of the two AI bots generates the persona and the other of the two AI bots executes the persona.

It will be understood that the disclosed system may implement both customizable personas and stackable knowledge expert resources.

It will also be understood that stackable knowledge expert resources may be accessed proactively in order to provide information to a user even without an inquiry from the user. For example, in some embodiments, a healthcoach feature may be configured to proactively direct a query to a first knowledge expert resource. Based on data received from the first knowledge expert resource, the system may then direct a second query to a second knowledge expert resource. Based on data received from the second knowledge expert resource, the system may provide information to a user.

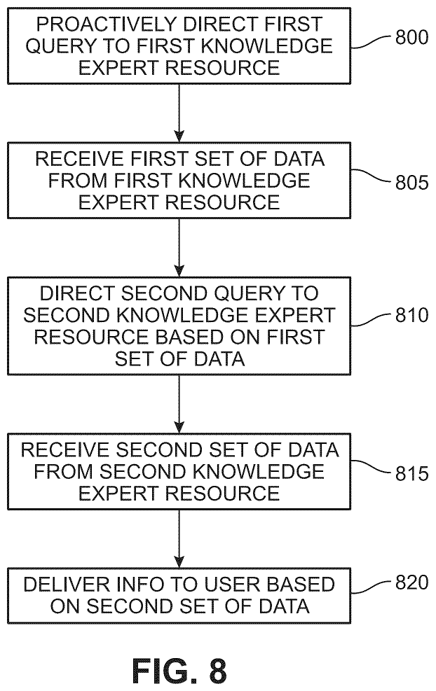

FIG. 8 is a schematic diagram illustrating a process of proactively retrieving data from multiple knowledge expert resources and providing information to a user based on the retrieved data. A virtual agent system may include a device processor and a non-transitory computer readable medium having stored thereon instructions, executable by the processor. The instructions may be for performing the steps illustrated in FIG. 8. In particular, the system may be configured to proactively direct a first query to a first knowledge expert resource based on the first request for information (800). In addition, the system may be configured to receive a first set of data from the first knowledge expert resource (805). Further, the system may be configured to direct a second query to a second knowledge expert resource based on the first set of data received from the first knowledge expert resource (810). Also, the system may be configured to receive a second set of data from the second knowledge expert resource (815). Finally, the system may deliver, to the user, information based on the second set of data (820).

The retrieval of data from the knowledge expert resources may be executed in a similar manner to that discussed above with respect to user inquiries. For example, the system may be configured to receive instructions for proactively retrieving data from one or more knowledge expert resources and providing information to the user based on the data. Also, at least one of the first knowledge expert resource and the second knowledge expert resource may be selected from a plurality of knowledge expert resources (see, e.g., FIG. 2). Further, it will be understood that the system may proactively consult with as many knowledge expert resources as necessary to retrieve the information to be provided to users. That is, any number of layers/tiers of knowledge expert resources may be accessed. In addition, the system may consult with knowledge expert resources in a serial manner, and the system may include predetermined instructions as to the order in which the knowledge expert resources will be consulted in order to retrieve information to be provided to users. Also, it will be noted that the information may be provided to the user via a text communication, audio speech communication, or video communication.

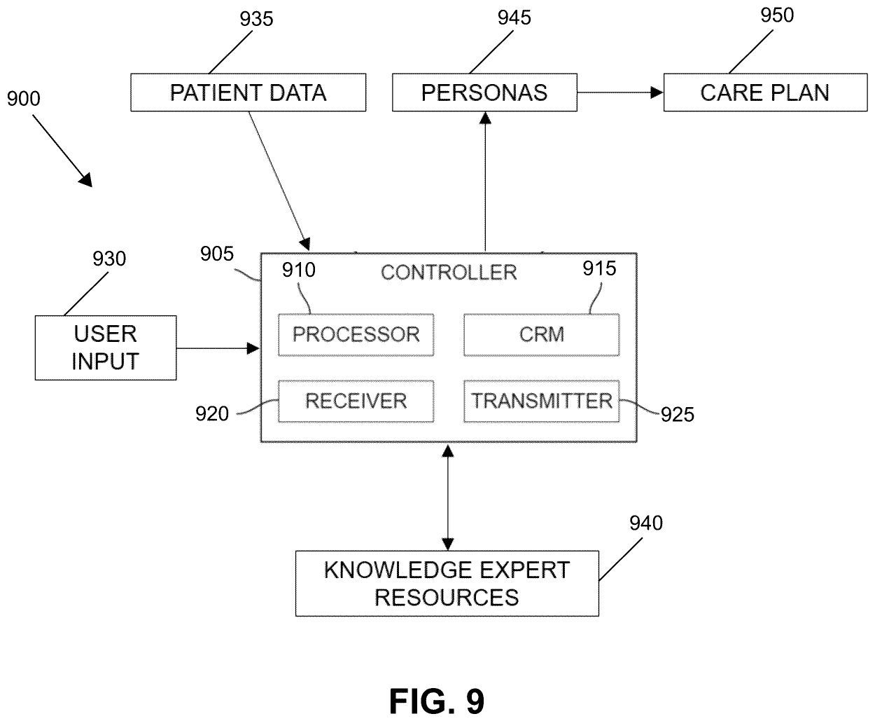

Additional personas and expert resources may also be implemented within the virtual agent to provide an AI-driven care planner. The care planner includes a schedule builder persona for creating personalized care plans and a message builder persona for crafting guideline compliant communications. FIG. 9 is a schematic block diagram of a system for generating personalized healthcare schedules within a virtual agent system. As shown in FIG. 9, a system 900 may include various computing and communications hardware. For example, as shown in FIG. 9, controller 905 may include a device processor 910 and a non-transitory computer readable medium 915 including instructions executable by device processor 910. Computer readable medium 915 may include any suitable computer readable medium, such as a memory, e.g., RAM, ROM, flash memory, or any other type of memory known in the art. Controller 905 may include other computing hardware, such as servers, integrated circuits, displays, etc.

Further, controller 905 may include networking hardware configured to interface with other nodes of a network, such as a LAN, WLAN, or other networks. For example, as shown in FIG. 9, controller 905 may include a receiver 920 and a transmitter 925. (It will be appreciated that, in some embodiments, the receiver and transmitter may be combined in a transceiver.) Receiver 920 and transmitter 925 may be configured to provide communication with other nodes of the system. Such communication may be executed via any suitable format, such as satellite communication, radiofrequency signals, etc.

Controller 905 may be provided at any suitable location. In some cases, controller 905 may be provided at a headquarters of a service provider. In other cases, controller 905 may be provided at a dedicated host facility configured to coordinate the virtual agent processes.

Computer readable medium 915 of controller 905 may include instructions for receiving a user request for one or more services. For example, as shown in FIG. 9, controller 905 may be configured to receive user input 935. For example, some users may access the system via the Internet, e.g., with a laptop. Some users may access the system via an application (app) on a personal electronic device, such as a smart phone. These users may submit requests for information using these access tools.

In addition, system 900 may be configured to receive patient data 935. Patient data 935 may include any medical and personal data may be received by controller 905 including, for example, medical records, demographic information, prescription drug information, etc.

Also, system 900 may consult one or more knowledge expert resources 940 to retrieve and analyze information. Exemplary knowledge expert resources 940 will be discussed in greater detail below.

System 900 is configured to implement multiple personas 945 which are each configured to provide a different aspect of the service for which system 900 is configured. Utilizing personas 945, system 900 provides a care plan 950, including a schedule of care and periodic messaging.

As noted above, system 900 can provide a schedule of care as part of care plan 950. System 900 may do this using a schedule builder persona. The schedule builder persona is responsible for creating personalized multi-week (e.g., 12 weeks) care plans for healthcare users. The schedule builder persona leverages user data, including health conditions, goals, and personality traits, to craft a tailored schedule of communications. The schedule builder persona works closely with a personality classification expert and a case management expert to ensure that the communication schedule aligns with the user's health needs and personality type. The output is a JSON-formatted schedule that includes dates, subjects, and message content sections (e.g., Welcome, Incentive Status, Personalized Wellbeing Tips) designed to maximize user engagement and interaction to achieve their health goals.

As also noted above, system 900 can provide periodic messages to the user. To do this, system 900 utilizes a message builder persona. The message builder persona generates personalized email messages based on the schedule provided by the schedule builder persona. The messages focus on encouraging users to complete their wellbeing activities and providing personalized health tips. The message builder persona relies on an incentive management expert for incentive status, the personality classification expert to tailor the message to the user's personality type, and the case management expert to tailor the wellbeing tips to the user's identified conditions. Each message includes a subject, welcome section, incentive status, personalized wellbeing tips, and a motivational message, ensuring that the content is compliant with healthcare regulations and effectively motivates the user.

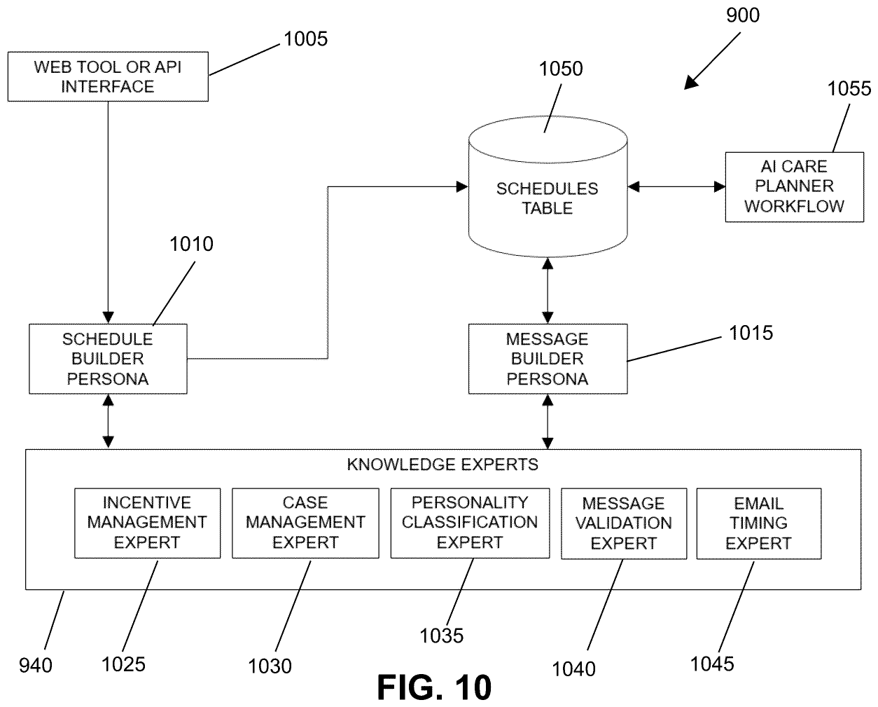

FIG. 10 is a more detailed block diagram of the system shown in FIG. 9. As shown in FIG. 10, system 900 may implement a web tool or application programming interface (API) 1005 by which a patient/user may interact with system 900. Based on the user's input at interface 1005 and received patient data, a schedule builder persona 1010 may initiate the construction of a care plan including a schedule of messaging.

Accordingly, a system for generating personalized healthcare schedules within a virtual agent system may include a device processor and a non-transitory computer readable medium having stored thereon instructions, executable by the processor, for receiving patient data regarding a patient and utilizing a schedule builder persona within the virtual agent system to create a care plan that is tailored to the patient's needs, has a predetermined duration, and includes details regarding timing and content of communication to be made with the patient.

In order to generate the care plan, schedule builder persona 1010 may be configured to consult with one or more knowledge expert resources (abbreviated as “knowledge experts” in the figures). For example, schedule builder persona 1010 may consult an incentive management expert resource 1025, a case management expert resource 1030, and a personality classification expert resource 1035.

The incentive management expert resource 1025 may be configured to monitor the user's incentive program progress and provide feedback on the steps needed to achieve their incentives. Incentive management expert resource 1025 integrates this information with schedule builder persona 1010 and a message builder persona 1015 to ensure that the user is consistently guided towards meeting their goals. Accordingly, the computer readable medium 915 (see FIG. 9) may further include instructions for consulting incentive management expert resource 1025 to incorporate user-specific goals and rewards into the care plan, ensuring alignment with health plan incentives of the patient.

Accordingly, the computer readable medium may further include instructions for consulting an incentive management expert resource to assess user wellbeing, receiving feedback from the incentive management expert resource, and customizing one or more of the communications of the series of personalized messages to provide guidance to the patient on how to achieve improved wellbeing or to meet certain incentives.

Case management expert resource 1030 is configured to develop guidelines for crafting patient messages based on presented conditions and risk analysis data. Case management expert resource 1030 offers useful information to assist the care planner in its task. Case management expert resource 1030 receives and analyzes information about a patient's risk level and medical conditions. This input is used by both schedule builder persona 1010 and message builder persona 1015 to tailor the communication schedule and message content, making the system more effective in engaging the user.

Accordingly, the computer readable medium may further include instructions for consulting a case management expert resource to develop guidelines for crafting patient messages based on presented conditions and risk analysis data, and customizing one or more of the communications of the series of personalized messages based on the guidelines developed by the case management expert resource.

Personality classification expert resource 1035 is configured to analyze the user's personality type using the TIPI 10-question personality test and provide insights on how to craft persuasive messages tailored to the user's personality. This input is used by both schedule builder persona 1010 and message builder persona 1015 to tailor the communication schedule and message content, making the system more effective in engaging the user.

As shown in FIG. 10, message builder persona 1015 may be configured to consult a message validation expert resource 1040 and an email timing expert resource 1045. Message validation expert resource 1040 ensures that all communications adhere to strict guidelines including, for example, HIPAA compliance. Additionally, email timing expert resource 1045 recommends optimal times for sending emails to maximize patient engagement.

Message builder persona 1015 is configured to query a schedules table 1050 and find messages that need to be built in the coming days. In addition, message builder persona 1015 gathers member data and feedback from the user utilizing a feedback collector persona, which will be discussed in great detail below. Further, message builder persona 1015 will consult with incentive management expert resource 1025. Finally, message builder persona 1015 builds a personalized email message to the user and saves the message back to schedules table 1050.

Once message builder persona 1015 generates messages and they are stored in schedules table 1050, system 900 will implement an AI care planner workflow 1055. As part of workflow 1055, system 900 will query schedules table 1050 for messages to send at the current date/time and send any scheduled messages to the user. In addition, as part of workflow 1055, system 900 will also post the messages to a message board of the user within system 900. It will also be noted that the messages may be sent via email, text message, or via any other suitable messaging platform.

Accordingly, the computer readable medium may further include instructions for utilizing message builder persona 1015 within the virtual agent system to generate a series of personalized messages based on the created care plan using message builder persona 1015. In addition, the computer readable medium may further include instructions for collecting data regarding the patient's personality type and generating one or more messages of the series of personalized messages that are customized according to the patient's personality type.

Also, the computer readable medium may further include instructions for consulting a message validation expert resource to ensure that all communications of the series of personalized messages adhere to predetermined guidelines and/or regulations. The computer readable medium may further include instructions for consulting an email timing expert resource and optimizing timing of the communications of the series of personalized messages to maximize engagement by the patient.

In addition, the computer readable medium may further include instructions for collecting data regarding a compliance status of the patient, reflecting the patient's compliance with one or more healthcare directives, and generating one or more updates to the care plan that are customized in view of the compliance status of the patient.

System 900 may also be configured to collect user feedback regarding the care plan and its messaging and update the care plan based on the feedback. In particular, system 900 may include a feedback collector persona configured to collect the feedback from the user. The feedback collector persona collects and analyzes user feedback, allowing the system to dynamically adjust care plans and suggest additional insurance products based on the user's expressed needs. This includes recommending specific products like drug membership programs when a user expresses concerns about medication costs. In addition, the feedback collector persona may receive and accommodate user's concerns regarding other healthcare topics, such as nutrition, allergy information, fitness and exercise, etc. This capability allows the AI care planner to not only react to user behavior but to proactively suggest tailored solutions, making the system more responsive and user-centric.

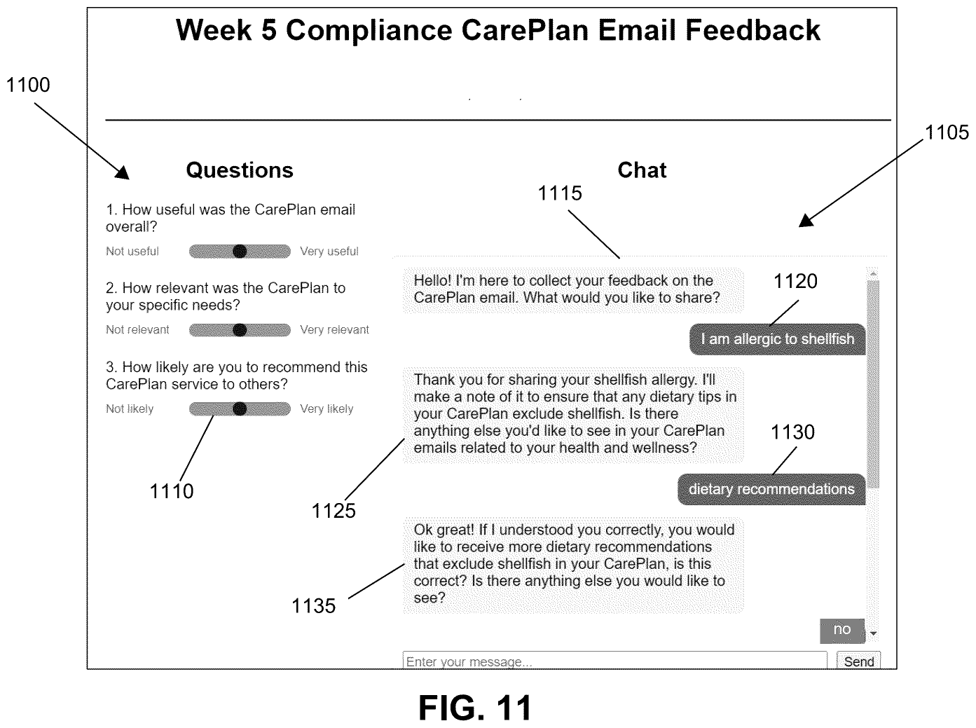

FIG. 11 is a schematic illustration of a feedback collection interface associated with a feedback collection persona utilized by the system of FIGS. 9 and 10. As shown in FIG. 11, the interface may include a series of questions 1100 as well as a chat interface 1105. In some embodiments, an input slider 1110 may be provided for the user to input their response to each of the questions 1100. It will be appreciated, however, that the interface may be configured to receive other types of input in response to the questions. Also, the interface shows three basic feedback questions, but could include any number of questions.

In addition, as also shown in FIG. 11, the feedback interface may include an AI chat interface 1105. The system may be configured to receive the user's input via a chat window 1115 and to provide real-time guidance and tips as well as make long-term updates to the user's care plan and the associated messaging. As shown in FIG. 11, the user may make an input 1120 about a food allergy, and the system response at 1125 by noting that the care plan will be updated accordingly. In addition, upon clarifying at 1130 that the user wishes to receive more information regarding dietary recommendations, the system considers the user's input collectively and responds at 1135 confirming that the user wishes to receive dietary recommendations that exclude the food to which they are allergic, in this case shellfish.

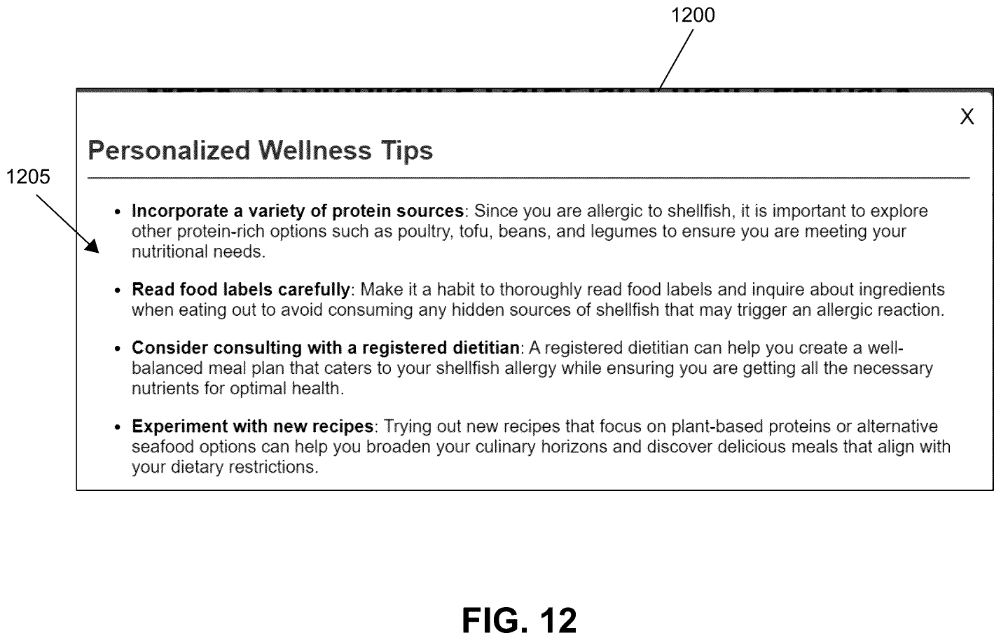

In addition to updating the care plan, the system may be configured to provide immediate personalized tips in view of the user's feedback in chat window 1115. FIG. 12 is a schematic illustration of a pop-up window providing personalized wellness tips based on user feedback received at the interface shown in FIG. 11. As shown in FIG. 12, a pop-up window 1200 may include a bullet list 1205 of tips that are generated based on the user's feedback. In particular, the list includes 4 tips regarding dietary recommendations taking into consideration the user's allergy to shellfish. Therefore, the system can provide both immediate recommendations via the pop-up window at the conclusion of the chat, as well as long-term recommendations and messaging via the care plan.

Accordingly, the computer readable medium may further include instructions for continuously updating the care plan based on real-time feedback received from the patient, as processed by a feedback collection persona. Also, the computer readable medium may further include instructions for providing immediate guidance to the patient upon receipt of the real-time feedback.

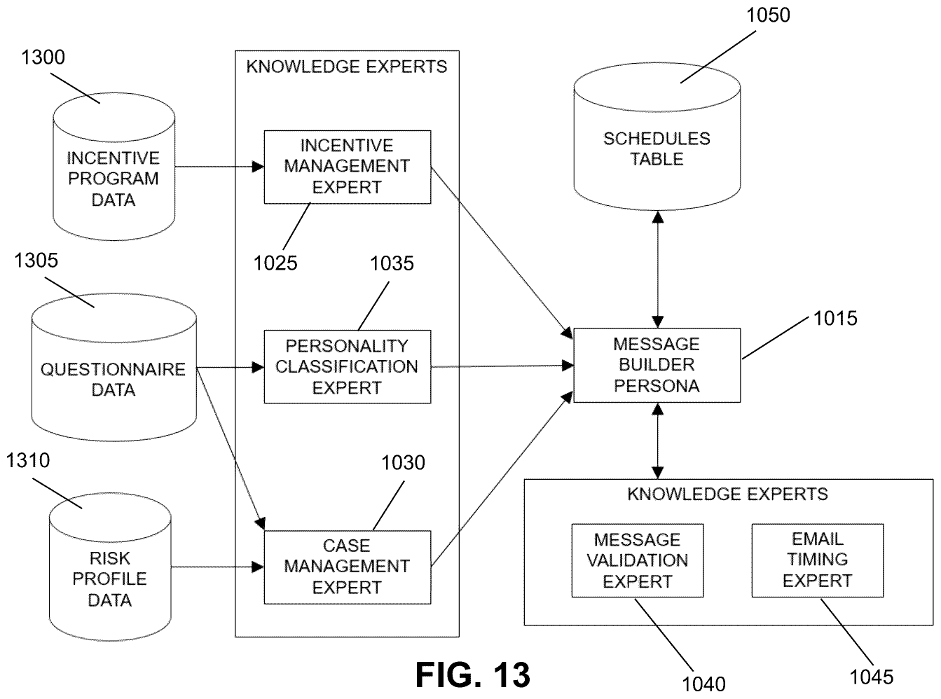

FIG. 13 is a schematic dataflow diagram illustrating the implementation of message builder persona. As shown in FIG. 13, the knowledge expert resources pull data from multiple repositories. For example, incentive management expert resource 1025 retrieves incentive program data 1300 regarding various incentives that may be relevant to the patient. In addition, questionnaire data 1305 may be considered by personality classification expert resource 1035 and case management expert resource 1030. Case management expert resource 1030 may also retrieve risk profile data 1310 for consideration.

As further shown in FIG. 13, message builder persona 1015 may consult incentive management expert resource 1025, personality classification expert resource 1035, and case management expert resource 1030, as well as message validation expert 1040 and email timing expert resource 1045. Once message builder persona 1015 has consulted the various expert resources, as discussed above, it generates one or more messages and uploads the messages to schedules table 1050.

In some embodiments, the disclosed system may also implement a healthcare program specialist persona, which may consider various healthcare (e.g., insurance) programs for the patient in question. The healthcare program specialist persona is configured to navigate various healthcare programs, such as drug insurance programs, and match patients with suitable options based on their current medical conditions and medications. This persona assists users in identifying potential programs, such as insurance programs, that could provide optimal coverage for their specific needs. For example, for prescription drug considerations, the healthcare program specialist persona analyzes the patient's drug regimen and health status in order to recommend appropriate insurance options, explaining the benefits and coverage details of each program. Additionally, the healthcare program specialist persona offers guidance through the enrollment process, helping users understand and complete necessary paperwork, meet eligibility requirements, and successfully join their chosen insurance program. By providing personalized assistance and expertise, this persona aims to ensure patients have access to the most beneficial and cost-effective drug coverage available to them.

This approach combines personalized healthcare guidance with targeted healthcare (e.g., insurance) program matching, streamlining a typically complex and overwhelming process for patients. By leveraging expert knowledge to analyze individual medical needs and insurance options simultaneously, it offers a uniquely tailored solution that can significantly improve patient access to necessary medications and reduce financial burden. Furthermore, the inclusion of enrollment assistance ensures that patients not only identify suitable programs but also successfully navigate the often daunting application process, increasing the likelihood of obtaining optimal program participation/enrollment (e.g., drug coverage) and ultimately enhancing their overall healthcare experience.

Accordingly, a system for directing a patient to healthcare programs within a virtual agent system may include a device processor and a non-transitory computer readable medium having stored thereon instructions, executable by the processor, for receiving patient data regarding a patient and utilizing a healthcare program specialist persona to navigate prescription drug programs and identify suitable options for the patient based on the patent's current medications and medical conditions.

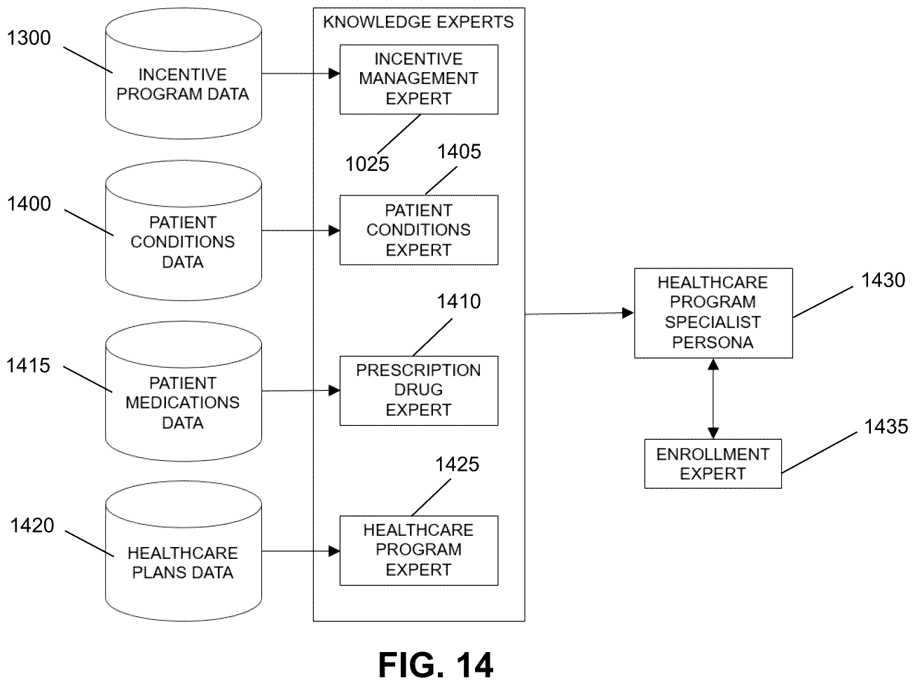

FIG. 14 is a schematic data workflow illustrating the implementation of a healthcare program specialist persona. As shown in FIG. 14, incentive management expert resource 1025, the functionality of which is discussed above, may receive incentive program data 1300. In addition, patient conditions data 1400 may be received by a patent conditions expert resource 1405. Similarly, a prescription drug expert resource 1410 may receive patient medications data 1415, and a healthcare program expert resource 1425 may receive healthcare plans data 1420.

Patient conditions expert resource 1405 may be configured to evaluate the received patient conditions data 1400, distill the data, and compile the data for consideration in conjunction with the other information and conclusions provided by other knowledge expert resources.

Prescription drug expert resource 1410 is configured to connect a member's medical conditions with appropriate prescription medications. This expert resource considers extensive pharmaceutical data and analyzes a patient's health profile and current medication regimen, ensuring that all prescribed drugs align effectively with the individual's conditions. The prescription drug expert resource's role is twofold: it conducts thorough checks on the member's current drug use to identify any potential issues or opportunities for optimization, and it also maintains vigilance over the member's prescription history across different healthcare programs. For example, this comprehensive approach allows the prescription drug expert resource to remind members if they have inadvertently continued purchasing medications through previous programs, potentially helping to avoid redundancy, reduce costs, and prevent complications from duplicate prescriptions. By providing this detailed oversight and personalized guidance, the prescription drug expert resource assists the patient by optimizing medication management and enhancing overall patient care.

Accordingly, the computer readable medium may further include instructions for consulting a prescription drug expert resource to ensure that all drugs prescribed to the patient optimally align with the user's conditions. Further, the computer readable medium may include instructions for consulting a prescription drug expert resource to monitor a prescription history of the patient across different healthcare programs and providing the patient with a message reminding the patient of their latest healthcare programs in the event the patient purchases medication through a healthcare program that the patient has replaced with a program in which the patient has more recently enrolled.

Healthcare program expert resource 1425 is configured to analyze and interpret complex healthcare plans, such as insurance plans, in order to match them with users' specific medical conditions and needs. This expert resource meticulously examines the details of various insurance options, including coverage limits, deductibles, copayments, and formularies, to determine how well each plan aligns with a user's particular health profile. By considering factors such as the user's current medications, ongoing treatments, and potential future healthcare needs, the expert resource provides a comprehensive assessment of which plans offer the most suitable and cost-effective coverage. This personalized approach helps users navigate the often confusing landscape of healthcare (e.g., insurance) options, enabling them to make informed decisions about their healthcare coverage. The healthcare program expert resource's insights can potentially lead to significant cost savings and improved healthcare outcomes by ensuring users select plans that best support their individual medical requirements.

Accordingly, the computer readable medium may further include instructions for consulting a healthcare program expert resource to analyze and interpret healthcare programs to match them with the patient's specific medical conditions and needs, and providing a recommendation to the patient for the patient to enroll in one or more new healthcare programs.

Thus, incentive management expert resource 1025, patient conditions expert resource 1405, prescription drug expert resource 1410 and healthcare program expert resource 1425 may provide analysis and data to a healthcare program specialist persona 1430, as shown in FIG. 14.

In addition, the system may further implement an enrollment expert resource 1435, which may be configured to assist users through the complex process of enrolling in various healthcare (e.g., insurance) products. This expert resource utilizes extensive information about different insurance offerings and their various intricacies, allowing it to provide clear, accurate information to users about the products they are considering. The role of the enrollment expert resource extends beyond merely facilitating enrollment; it serves as a knowledgeable resource, capable of providing answers to a wide range of questions about coverage details, benefits, limitations, and procedures specific to each insurance product. By combining technical knowledge of enrollment processes with comprehensive product understanding, this expert resource helps users navigate potential obstacles, clarify confusing terms or conditions, and make well-informed decisions about their insurance choices. The enrollment expert resource provides guidance that aims to streamline the often daunting task of insurance enrollment, ensuring users feel confident and well-supported throughout the entire process. As an example of the enrollment expert resource's capabilities, in some embodiments, the enrollment expert resource may be configured to check that the patient is receiving their drugs through the drug membership program in which they are enrolled.

Accordingly, the computer readable medium may further include instructions for consulting an enrollment expert resource in order to facilitate enrollment in one or more healthcare programs.

In some embodiments, the system may be configured to redirect user interaction through multiple apps using various application programming interfaces (APIs). For example, the system may redirect a user to an external API for purposes of enrollment in a new healthcare (insurance) program. Similarly, the system may redirect a user to an external API for purposes of scheduling a new appointment with a doctor.

Accordingly, the computer readable medium also includes instructions for receiving prescription drug data and medical conditions data associated with the patient and redirecting a user of the virtual agent to one or more applications external to the virtual agent such that the user interacts with one or more application programming interfaces (APIs) associated with the one or more external applications to modify at least one aspect of the patient's healthcare.

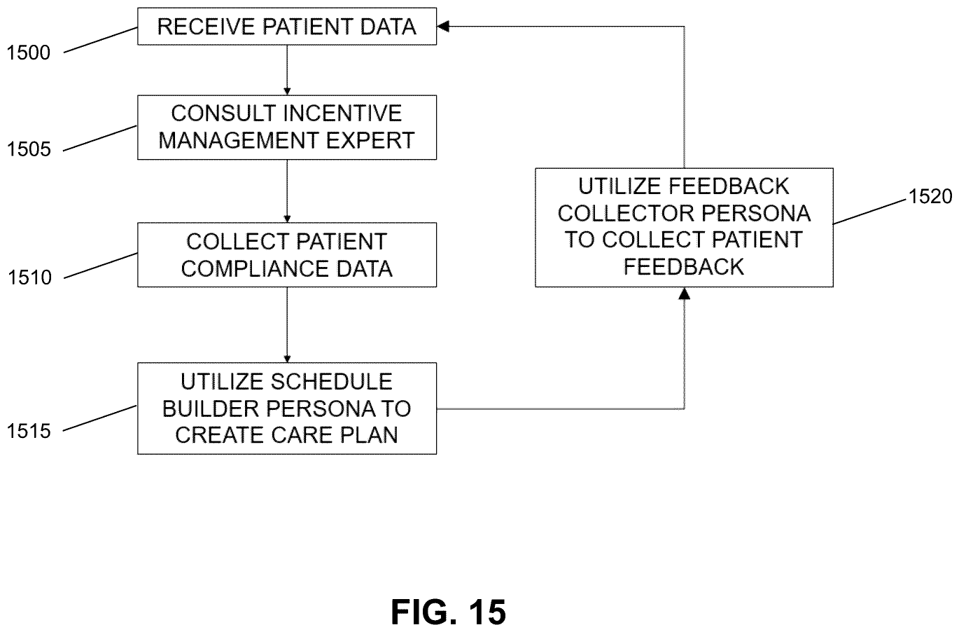

FIG. 15 is a flowchart illustrating a method of implementing a schedule builder persona. As shown in FIG. 15, patient data may be received at an initial step 1500. Next, the system may consult an incentive management expert resource at step 1505. At step 1510, the system may collect patient compliance data. Once these steps have been completed, the system may, at step 1515, utilize the schedule builder persona to create a care plan. Further, at step 1520, the system may utilize the feedback collector persona to collect feedback from the patient. The system may then consider all of the collected data, including he patient feedback, and update the care plan using the schedule builder persona. In some embodiments, the system may immediately base updates to the care plan on patient feedback without passing through the rest of the process.

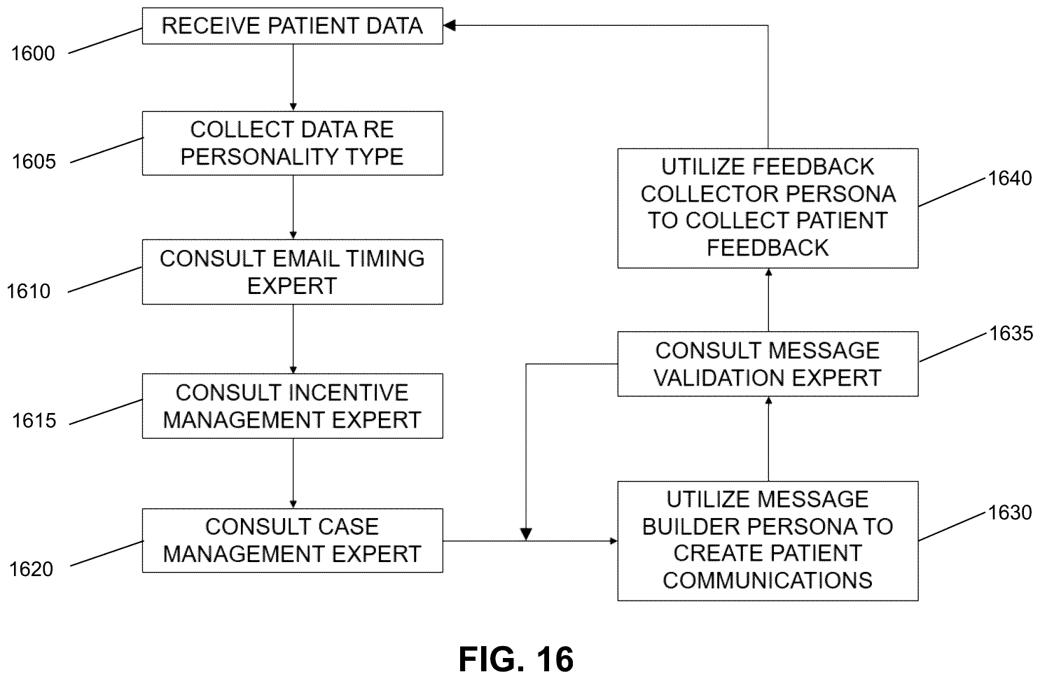

FIG. 16 is a flowchart illustrating a method of implementing a message builder persona. As shown in FIG. 16, patient data may be received at an initial step 1600. In addition, at step 1605, data may be collected regarding the user's personality type. At step 1610, the system may consult the email timing expert. Further, at step 1615, the system may consult the incentive management expert, and at step 1620 may consult the case management expert. It will be understood that this collection of data and consultation of various experts may occur in a different order or simultaneously or partially simultaneously. That is, certain consultations may occur within the system simultaneously. With all the data received and collected, the system may, at step 1630, utilize the message builder persona to create patient communications.

Once the patient communications have been generated, the system, at step 1635, may consult the message validation expert resource. If any of the generated messages do not adhere to the predetermined guidelines and/or regulations, the process loops back and the system regenerates the communications ensuring compliance with all relevant guidelines and regulations.

Also, similar to discussed above with respect to FIG. 15, the process of FIG. 16 may also include, at step 1640, the utilization of the feedback collector persona to collect patient feedback, which may then be fed back into the process for further consideration and updating the patient communications accordingly.

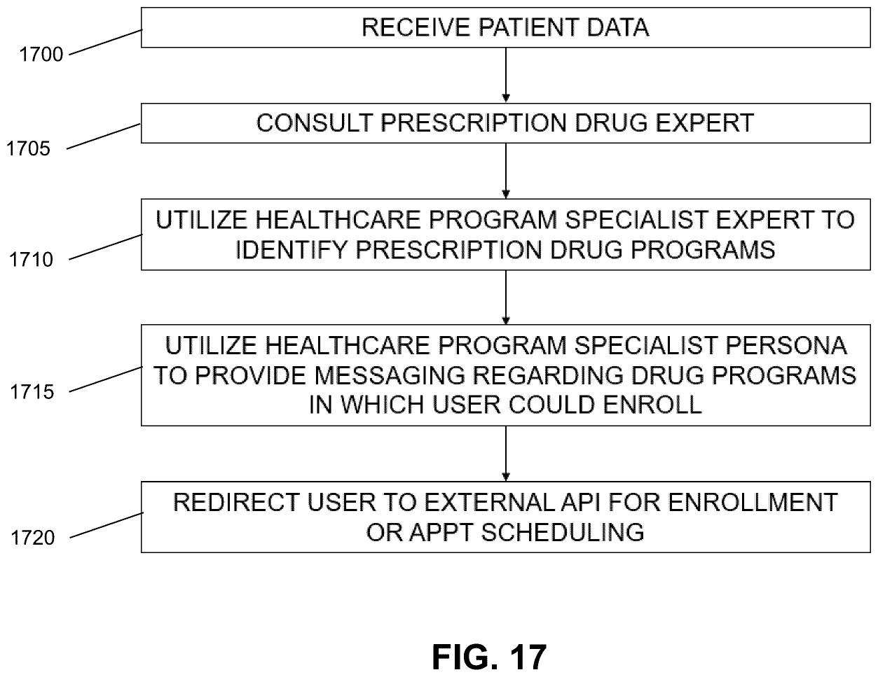

FIG. 17 is a flowchart illustrating a method of implementing a healthcare program specialist persona. As shown in FIG. 17, patient data may be received at an initial step 1700. In addition, at step 1705, the system may consult the prescription drug expert resource. With this data and information compiled from these sources, the system may then, at step 1710, utilize the healthcare program specialist persona to identify prescription drug programs that may be beneficial to the user.

Once the system has identified prescription drug programs that may be beneficial to the user, the system may, at step 1715, utilize the healthcare program specialist persona to provide messaging regarding drug programs in which the user could enroll. Also, at step 1720, the system may redirect the user to one or more external APIs, for example, for program enrollment or appointment scheduling.

Further, in some embodiments, the system may provide messaging regarding the adherence to drug programs in which the patient is already enrolled. For example, if the patient is enrolled in one prescription drug insurance program that is relatively expensive, and the system identifies and enrolls the patient in a less expensive program, if the user then purchases medication through the old (expensive) program, the system may provide a reminder to the user to utilize the less expensive program.

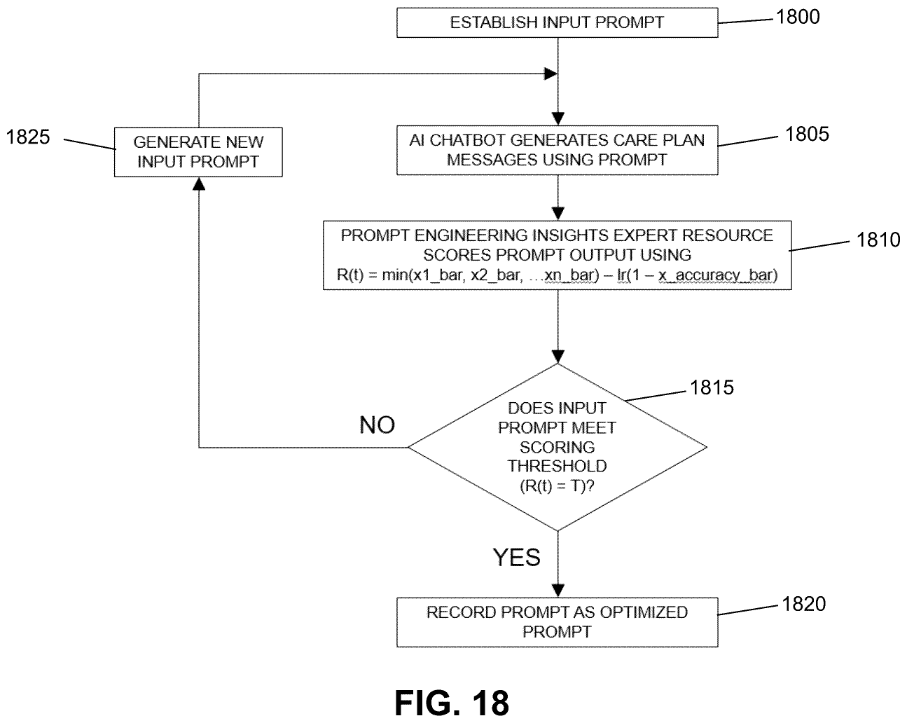

As an alternative to deep Q learning, in some embodiments, the disclosed virtual agent system may utilize a self-learning generative AI prompt optimization process. FIG. 18 is a schematic diagram of an exemplary embodiment of a self-learning generative AI prompt optimization process. As shown in FIG. 18, the system may be configured to establish an input prompt at step 1800. Then, at step 1805, the AI chatbot may generate care plan messages using the established prompt.

At step 1810, the system may utilize a prompt engineering insights expert resource to score the prompt output. In some embodiments, the system may score the prompt output based on various data regarding, for example, the usefulness of the information (which may be referred to as variable x1), the accuracy of the information (x2), the overall friendliness of the persona (x3), and/or any other suitable data. Accordingly, the prompt output score R(t) may be calculated based on the following formula: R(t)=min(x1_bar, x2_bar, . . . xn_bar)−Ir(1−x_accuracy_bar), where “Ir” is the learning rate.

At step 1815, the system may then determine whether the input prompt meets a scoring threshold T. In other words, the system asks whether R(t)=T. If so, then the system records the established prompt as an optimized prompt at step 1820 and proceeds to keep using it. If not, then the system generates a new input prompt at step 1825 and returns to step 1805 where the AI chatbot generates care plan messages using the new input prompt and the procedure continues from there as previously discussed.

The system may also include an output manager expert resource configured to generate omnichannel communications in real time, such as emails, interactive web pages, PDF export files, mobile device notifications, API call into external web services, etc. Whenever the user is interacting with the AI chatbots described above, the output manager expert resource may be implemented to generate the omnichannel communications. Thes omnichannel communications may take various forms. In some cases, the communications may include a PDF claim form, which may have been at least partially filled out by the system and sent to the user. In some cases, the communications may include a URL directing the user to an interactive feedback interface webpage. In still other cases, the communications may pass along suggestions and/or advertisements for various healthcare products based on the user's interaction with the system and their medical information.

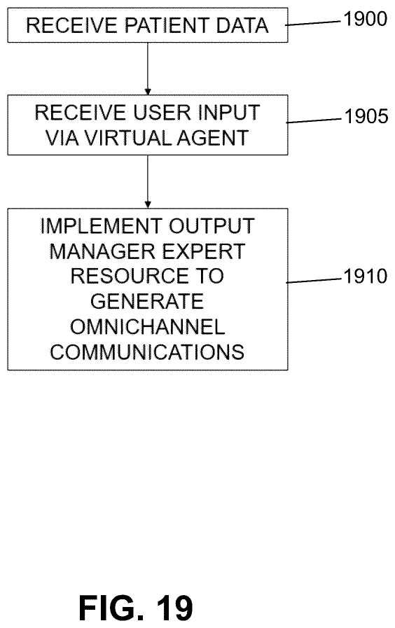

Accordingly, the computer readable medium may be configured to execute a method of omnichannel communication. FIG. 19 is a flowchart illustrating a method of omnichannel communication. As shown in FIG. 19, the method may include receiving patient data at step 1900 and receiving user input via a virtual agent at step 1905. Further, the method may include, based on the received patient data and user input, implementing an output manager expert resource to generate a plurality of omnichannel communications to be sent to the patient (step 1910).

It may be appreciated that the steps of the various processes discussed above could be performed in different orders in some other embodiments. In addition, it will be appreciated that the consultation of expert resources may be stacked. That is, the system may consult a first expert resource and then consult a second expert resource based on the consultation of the first expert resource.

While various embodiments have been described, the description is intended to be exemplary, rather than limiting, and it will be apparent to those of ordinary skill in the art that many more embodiments and implementations are possible that are within the scope of the embodiments. Although many possible combinations of features are shown in the accompanying figures and discussed in this detailed description, many other combinations of the disclosed features are possible. Any feature of any embodiment may be used in combination with or substituted for any other feature or element in any other embodiment unless specifically restricted. Therefore, it will be understood that any of the features shown and/or discussed in the present disclosure may be implemented together in any suitable combination. Accordingly, the embodiments are not to be restricted except in light of the attached claims and their equivalents. Also, various modifications and changes may be made within the scope of the attached claims.

We claim:

1. A system for generating personalized healthcare schedules within a virtual agent system, comprising:
a device processor; and
a non-transitory computer readable medium having stored thereon instructions, executable by the processor, for performing the following steps:
receiving patient data regarding a patient; and
utilizing a schedule builder persona within the virtual agent system to create a care plan that is tailored to the patient's needs, has a predetermined duration, and includes details regarding timing and content of communication to be made with the patient.

2. The system of claim 1, wherein the computer readable medium further includes instructions for consulting an incentive management expert resource to incorporate user-specific goals and rewards into the care plan, ensuring alignment with health plan incentives of the patient.

3. The system of claim 1, wherein the computer readable medium further includes instructions for continuously updating the care plan based on real-time feedback received from the patient, as processed by a feedback collection persona.

4. The system of claim 3, wherein the computer readable medium further includes instructions for providing immediate guidance to the patient upon receipt of the real-time feedback.

5. The system of claim 1, wherein the computer readable medium further includes instructions for collecting data regarding a compliance status of the patient, reflecting the patient's compliance with one or more healthcare directives; and
generating one or more updates to the care plan that are customized in view of the compliance status of the patient.

6. A system for generating personalized healthcare messaging within a virtual agent system, comprising:
a device processor; and
a non-transitory computer readable medium having stored thereon instructions, executable by the processor, for performing the following steps:
receiving patient data regarding a patient; and
utilizing a message builder persona within the virtual agent system to generate a series of personalized messages based on the created care plan.

7. The system of claim 6, wherein the computer readable medium further includes instructions for collecting data regarding the patient's personality type; and
generating one or more messages of the series of personalized messages that are customized according to the patient's personality type.

8. The system of claim 6, wherein the computer readable medium further includes instructions for consulting a message validation expert resource to ensure that all communications of the series of personalized messages adhere to predetermined guidelines and/or regulations.

9. The system of claim 6, wherein the computer readable medium further includes instructions for consulting an email timing expert resource; and
optimizing timing of the communications of the series of personalized messages to maximize engagement by the patient.

10. The system of claim 6, wherein the computer readable medium further includes instructions for consulting an incentive management expert resource to assess user wellbeing;
receiving feedback from the incentive management expert resource; and
customizing one or more of the communications of the series of personalized messages to provide guidance to the patient on how to achieve improved wellbeing or to meet certain incentives.

11. The system of claim 6, wherein the computer readable medium further includes instructions for consulting a case management expert resource to develop guidelines for crafting patient messages based on presented conditions and risk analysis data; and
customizing one or more of the communications of the series of personalized messages based on the guidelines developed by the case management expert resource.

12. A system for directing a patient to healthcare programs within a virtual agent system, comprising:
a device processor; and
a non-transitory computer readable medium having stored thereon instructions, executable by the processor, for performing the following steps:
receiving patient data regarding a patient; and
utilizing an healthcare program specialist persona to navigate prescription drug programs and identify suitable options for the patient based on the patent's current medications and medical conditions.

13. The system of claim 12, wherein the computer readable medium further includes instructions for consulting a prescription drug expert resource to ensure that all drugs prescribed to the patient optimally align with the user's conditions.

14. The system of claim 12, wherein the computer readable medium further includes instructions for consulting a prescription drug expert resource to monitor a prescription history of the patient across different healthcare programs; and
providing the patient with a message reminding the patient of their latest healthcare programs in the event the patient purchases medication through a healthcare program that the patient has replaced with a program in which the patient has more recently enrolled.

15. The system of claim 12, wherein the computer readable medium further includes instructions for consulting a healthcare program expert resource to analyze and interpret healthcare programs to match them with the patient's specific medical conditions and needs; and
providing a recommendation to the patient for the patient to enroll in one or more new healthcare programs.

16. The system of claim 12, wherein the computer readable medium further includes instructions for consulting an enrollment expert resource in order to facilitate enrollment in one or more healthcare programs.

17. The system of claim 12, wherein the computer readable medium further includes instructions for receiving prescription drug data and medical conditions data associated with the patient; and
redirecting a user of the virtual agent to one or more applications external to the virtual agent such that the user interacts with one or more application programming interfaces (APIs) associated with the one or more external applications to modify at least one aspect of the patient's healthcare.

18. A system for automated healthcare management of a patient, comprising:
a virtual agent system that includes multiple specialized personas, each configured to perform specific tasks within a healthcare management process, including:
a schedule builder persona configured to generate a personalized care plan based on patient data, integrating input from one or more expert resources to tailor the care plan to the patient's needs; and
a message builder persona configured to generate and schedule patient communications based on the generated personalized care plan, ensuring that each message is compliant with healthcare regulations;
the system being configured to consult multiple specialized expert resources for analysis of patient data and available healthcare programming, including:
a feedback expert resource configured to receive and analyze real-time feedback from the patient and provide insights that allow the schedule builder persona and message builder persona to adjust the generated care plan and the generated patient communications; and
an incentive management expert resource configured to monitor patient compliance with health plan activities set forth in the generated care plan and incorporating user-specific goals and rewards into the care plan.

19. The system of claim 18, wherein the system is further configured to collect data regarding the patient's personality type; and
generate one or more of the patient communications that are customized according to the patient's personality type.

20. The system of claim 18, wherein the system is configured to consult a message validation expert resource to ensure that all of the patient communications adhere to predetermined guidelines and/or regulations.

21. A method of self-learning generative artificial intelligence (AI) prompt optimization, comprising:
utilizing a device processor to execute instructions stored on a non-transitory computer readable medium for performing the following steps:
establishing an input prompt;
generating care plan messages using the input prompt;
using a prompt engineering insights expert resource to determine a prompt output score;
determining whether the prompt output score meets a predetermined scoring threshold; and
recording the input prompt as an optimized prompt if the prompt output score meets the predetermined scoring threshold and generating a new input prompt if the prompt output score does not meet the predetermined scoring threshold.

22. A method of omnichannel communication, comprising:
a utilizing a device processor to execute instructions stored on a non-transitory computer readable medium for performing the following steps:
receiving patient data regarding a patient;
receiving user input from the patient via a virtual agent; and
based on the received patient data and user input, implementing an output manager expert resource to generate a plurality of omnichannel communications to be sent to the patient.

23. The method of claim 22, wherein the omnichannel communications include one or more of the following: email messages, URL's to interactive web pages, PDF export files, mobile device notifications, and application programming interface (API) calls into external web services.
